(19) United States
(12) Patent Application Publication
(10) Pub. No.: US 2026/0216014 A1
(43) Pub. Date: Jul. 30, 2026

(54) Macroscopically Homogeneous Cosmetic Composition with an Aqueous Phase and an Oily Phase, a Volatile Hydrocarbon Oil and a Silicone Elastomer Containing Carboxylic Acid Functions

(71) Applicant: L'OREAL, PARIS (FR)

(72) Inventors: GUILLAUME KERGOSIEN, CHEVILLY-LARUE (FR); KÉVIN DU, CHEVILLY-LARUE (FR)

(73) Assignee: L'OREAL, Paris (FR)

(21) Appl. No.: 19/145,533

(22) PCT Filed: Dec. 13, 2023
(86) PCT No.: PCT/EP2023/085664

§ 371 Date: Jul. 3, 2025

(30) Foreign Application Priority Data
Jan. 4, 2023 (FR) FR2300058

(51) Int. Cl. A61K 8/06; A61K 8/31; A61K 8/892; A61K 8/898; A61Q 1/08; A61Q 1/10; A61Q 19/00

(52) U.S. Cl. CPC A61K 8/062; A61K 8/31; A61K 8/892; A61K 8/898; A61Q 1/08; A61Q 1/10; A61Q 19/00; A61K 2800/54; A61K 2800/805

(57) ABSTRACT

The present invention relates to a composition for caring for and/or making up keratin materials, in particular the skin, comprising, preferably in a physiologically acceptable medium: a) an oily phase comprising at least one volatile hydrocarbon oil H1; and b) an aqueous phase; and c) at least one silicone elastomer containing carboxylic acid functions; said composition having a product of the weight ratios [A]×[B] ranging from 0.07 to 0.21 with [A] denoting the weight ratio of the amount of silicone elastomer relative to the total weight of the composition to the total amount of volatile hydrocarbon oil(s) relative to the total weight of the composition; [B] denoting the weight ratio of the amount of aqueous phase relative to the total weight of the composition; said oily and aqueous phases forming therein a macroscopically homogeneous mixture.

### TECHNICAL FIELD

The present invention aims to provide, for the field of caring for and/or making up keratin materials, in particular the skin, a new macroscopically homogeneous composition comprising a) an oily phase comprising at least one volatile hydrocarbon oil, b) an aqueous phase and c) at least one silicone elastomer containing carboxylic acid functions.

Cosmetic makeup compositions are commonly used to give an aesthetic colour to keratin materials such as the skin, but also to enhance the beauty of irregular skin, by making it possible to hide marks and dyschromias, and to reduce the visibility of relief imperfections such as pores and wrinkles and also spots and acne marks.

Consumers are also looking for care and/or makeup products for keratin materials, in particular the skin, which have a texture that is comfortable from a sensory point of view and on application, in particular which are easy to take up and which spread easily over the area of keratin material to be covered.

Compositions comprising an oily phase and an aqueous phase forming a macroscopically homogeneous mixture (the distinction between the two mutually immiscible phases being impossible to see with the naked eye) such as oil/water emulsions, water-in-oil emulsions or bigels are galenic forms that are highly sought after by consumers for their creamy or gelled texture, their ease of application to the skin and their sensoriality in cosmetics, such as freshness, in the care field, in particular in anti-sun compositions, in anti-ageing compositions and in makeup such as foundations.

Published documents WO2015066161 (US20160200876), WO2015066165, WO2015066199, WO2015167963, WO20180165434 (U.S. Pat. No. 10,918,587B2), IPCOM000250448D and IPCOM000250657D have already proposed makeup compositions based on silicone elastomers containing carboxylic acid functions exhibiting good performance in terms of wear property and durability. However, these polymers generally have a texture which is difficult to take up and apply and have a tendency to give makeup compositions of which the consistency does not make it possible to take up and spread the product easily on the skin. When they are used in compositions based on an oily phase and an aqueous phase forming a macroscopically homogeneous mixture such as emulsions, these silicone elastomers tend to produce formulations which are unstable over time and for which phase separation is observed.

The objective of this invention is to produce a macroscopically homogeneous care and/or makeup composition comprising an oily phase and an aqueous phase and comprising at least one silicone elastomer containing carboxylic acid functions, which is stable, has a consistency that is sufficient, flexible and homogeneous so as to be taken up and easily spread on the skin and which has good sensory properties such as freshness and lightness during application.

In addition, the applicant has observed that the composition according to the invention also makes it possible to obtain a water-releasing effect during application to the skin with the formation of droplets during spreading, with also good stability over time.

In the course of its research, the applicant has discovered unexpectedly that this objective can be achieved with a composition for caring for and/or making up keratin materials, in particular the skin, comprising, preferably in a physiologically acceptable medium:
a) an oily phase comprising at least one volatile hydrocarbon oil H₁; and
b) an aqueous phase; and
c) at least one silicone elastomer containing carboxylic acid functions;
said composition having a product of the weight ratios [A]×[B] ranging from 0.07 to 0.21 with
[A] denoting the weight ratio of the amount of silicone elastomer relative to the total weight of the composition to the total amount of volatile hydrocarbon oil(s) relative to the total weight of the composition;
[B] denoting the weight ratio of the amount of aqueous phase relative to the total weight of the composition;
said oily and aqueous phases forming therein a macroscopically homogeneous mixture.

The compositions according to the invention are stable over time and have a consistency that is sufficient, flexible and homogeneous so as to be taken up and easily spread on the skin. They also have a feeling of freshness and lightness during application.

This discovery forms the basis of the invention.

The present invention relates to a composition for caring for and/or making up keratin materials, in particular the skin, comprising, preferably in a physiologically acceptable medium:
a) an oily phase comprising at least one volatile hydrocarbon oil H₁; and
b) an aqueous phase; and
c) at least one silicone elastomer containing carboxylic acid functions;
said composition having a product of the weight ratios [A]×[B] ranging from 0.07 to 0.21 with
[A] denoting the weight ratio of the amount of silicone elastomer relative to the total weight of the composition to the total amount of volatile hydrocarbon oil(s) relative to the total weight of the composition;
[B] denoting the weight ratio of the amount of aqueous phase relative to the total weight of the composition;
said oily and aqueous phases forming therein a macroscopically homogeneous mixture.

The invention also relates to a process for coating keratin materials, more particularly for making up and/or caring for keratin materials, such as the skin, characterized in that it comprises the application to keratin materials of an emulsion as defined above.

The invention more particularly relates to a process for making up and/or caring for the skin, characterized in that it comprises the application to the skin of a composition as defined above.

### DEFINITIONS

In the context of the present invention, the term “keratin materials” means the skin and more particularly the areas such as the face, the cheeks, the hands, the body, the legs and thighs, the area around the eyes, and the eyelids.

The term “physiologically acceptable” means compatible with the skin and/or its integuments, which has a pleasant colour, odour and feel, and which does not cause any unacceptable discomfort (stinging or tautness) liable to discourage the consumer from using this composition.

For the oily and aqueous phases of the composition of the invention, the expression “that they form a macroscopically homogeneous mixture” is intended to mean the fact that observation of the composition with the eye does not make it possible to distinguish the presence of the two mutually immiscible phases. The identification of each of the two phases, and the arrangement and distribution thereof within the composition can generally be observed only under a microscope.

### Oily Phase

The composition according to the invention for caring for and/or making up keratin materials comprises at least one fatty phase comprising at least one volatile hydrocarbon oil H₁.

The term “oil” refers to a fatty substance that is liquid at ambient temperature (25° C.) and atmospheric pressure (760 mmHg, i.e. 105 Pa).

The term “oily phase” refers to an organic phase that is liquid at ambient temperature (25° C.) and atmospheric pressure containing at least one oil and optionally the cosmetic additives which are soluble or miscible in said phase.

The term “hydrocarbon oil” refers to an oil mainly containing carbon and hydrogen atoms and possibly one or more functions chosen from hydroxyl, ester, ether and carboxylic functions.

For the purposes of the invention, the term “volatile oil” refers to any oil that is capable of evaporating on contact with the skin in less than one hour, at ambient temperature and atmospheric pressure. The volatile oil is a volatile cosmetic compound, liquid at ambient temperature, having in particular a non-zero vapour pressure, at ambient temperature and atmospheric pressure, in particular having a vapour pressure ranging from 2.66 Pa to 40 000 Pa, especially ranging from 2.66 Pa to 13 000 Pa and more particularly ranging from 2.66 Pa to 1300 Pa.

Examples of volatile hydrocarbon oils H₁ which can be used in the oily phase of the composition of the invention include hydrocarbon oils having from 8 to 16 carbon atoms, and in particular C₈-C₁₆ isoalkanes of petroleum origin (also called isoparaffins) such as isododecane (also called 2,2,4,4,6-pentamethylheptane), isodecane and isohexadecane. Mention may also be made of the oils sold under the trade names Isopar® or Permethyl®, branched C₈-C₁₆ esters, isohexyl neopentanoate, and mixtures thereof. Other volatile hydrocarbon oils, for instance petroleum distillates, notably those sold under the name Shell Solt by Shell, may also be used.

The volatile hydrocarbon oils H₁ which can be used in the compositions according to the invention can be chosen from volatile linear alkanes comprising from 6 to 14 carbon atoms.

Mention may be made, by way of examples of linear alkanes suitable for the invention, of the alkanes described in the patent applications WO 2007/068371 and WO 2008/155059 of Cognis (mixtures of different alkanes differing by at least one carbon). These alkanes are obtained from fatty alcohols, themselves obtained from coconut oil or palm oil.

Mention may be made, by way of examples of linear C₆-C₁₄ alkanes suitable for the invention, of n-hexane (C₆), n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and mixtures thereof.

Mention may notably be made of n-dodecane (C₁₂) and n-tetradecane (C₁₄) sold by Sasol under the references, respectively, Parafol 12-97® and Parafol 14-97®, and also mixtures thereof.

According to another embodiment, a mixture of n-dodecane and of n-tetradecane is used. It is in particular possible to use the dodecane/tetradecane mixture in the 85/15 weight ratio sold by Biosynthis under the reference Vegelight 1214®.

According to yet another embodiment, use is made of a mixture of volatile linear C₉-C₁₂ alkanes with the INCI name: C₉₋₁₂ Alkane, such as the product sold by the company Biosynthis under the reference Vegelight Silk®.

According to yet another embodiment, use is made of a mixture of n-undecane (C₁₁) and of n-tridecane (C₁₃), like those obtained in examples 1 and 2 of the application WO 2008/155059 from Cognis and like that sold under the trade name Cetiol Ultimate® by BASF.

According to one particularly preferred embodiment, the volatile hydrocarbon oil H₁ will be chosen from C₈-C₁₆ isoalkanes of petroleum origin, and more particularly isododecane.

According to a preferential embodiment of the invention, the oily phase is present in the composition in an amount ranging from 5% to 95% by weight, and more preferentially from 10% to 90% by weight relative to the total weight of the composition.

### Aqueous Phase

The composition of the invention comprises an aqueous phase. The latter forms a macroscopically homogeneous mixture with the oily phase.

The aqueous phase comprises water and optionally water-soluble or water-miscible ingredients, such as water-soluble solvents.

A water suitable for the invention can be a floral water, such as cornflower water, and/or a mineral water, such as Vittel water, Lucas water or La Roche-Posay water, and/or a thermal water.

In the present invention, the term “water-soluble solvent” denotes a compound which is liquid at ambient temperature and water-miscible (miscibility in water of greater than 50% by weight at 25° C. and atmospheric pressure).

The water-soluble solvents which can be used in the composition of the invention can additionally be volatile.

Mention may in particular be made, among the water-soluble solvents which can be used in the composition in accordance with the invention, of C₂-C₄ monoalcohols such as ethanol and isopropanol, glycols having from 2 to 8 carbon atoms, such as ethylene glycol, propylene glycol, 1,3-butylene glycol, propanediol, pentylene glycol, glycerol and dipropylene glycol, C₃-C₄ ketones and C₂-C₄ aldehydes.

According to a preferential embodiment, the weight ratio of the total amount of volatile hydrocarbon oil(s) relative to the total weight of the composition to the amount of aqueous phase relative to the total weight of the composition is greater than 0.085.

According to one particular form, the water is present in a concentration ranging from 5% to 95% by weight, and more particularly from 10% to 90% by weight, relative to the total weight of said composition.

According to one particular form, the aqueous phase of the composition is present in a concentration ranging from 5% to 95% by weight, and more particularly from 10% to 90% by weight, relative to the total weight of said composition.

According to a particular form, the non-solid compositions of the invention comprising an aqueous phase can be provided in various galenic forms such as
emulsions such as oil-water or water-oil emulsions in which one of the two oily and aqueous phases is dispersed in the other phase, known as continuous, in the form of droplets and the composition is macroscopically homogeneous to the naked eye;
bigels which are macroscopically homogeneous to the naked eye.

### Silicone Elastomer Containing Carboxylic Acid Functions

The composition according to the invention for caring for and/or making up keratin materials comprises b) at least one silicone elastomer containing carboxylic acid functions.

The term “organopolysiloxane elastomer” means a supple, deformable organopolysiloxane that has viscoelastic properties and especially the consistency of a sponge or a supple sphere. Its modulus of elasticity is such that this material withstands deformation and has limited stretchability and contractibility. This material is capable of regaining its original shape after stretching. In general, it is partially or completely crosslinked and is not cyclic.

The term “silicone elastomer containing carboxylic acid functions” means an organopolysiloxane elastomer comprising in its structure at least two carboxylic acid groups with at least one-COOH function.

The composition according to the invention advantageously has a content of active material of silicone elastomer containing carboxylic acid functions ranging from 2% to 25% by weight relative to the weight of the composition. Preferably, this content ranges from 5% to 20% by weight relative to the weight of the composition.

The silicone elastomers containing carboxylic acid functions that can be used in the compositions of the invention can be chosen from those described in published documents WO2015066161 (US20160200876), WO2015066165, WO2015066199, WO2015167963, WO20180165434 (U.S. Pat. No. 10,918,587B2), IPCOM000250448D, and IPCOM000250657D.

According to one particular embodiment, the silicone elastomer containing carboxylic acid functions is chosen from those corresponding to formula (I) below:

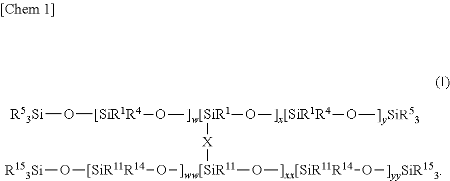

in which
the radical X corresponds to formula (i) or formula (i′) below:

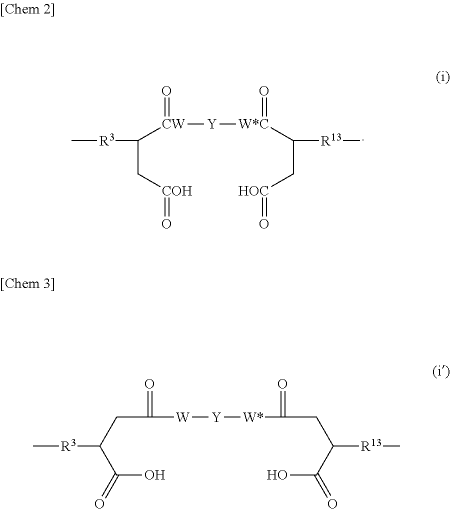

in which
W and W* independently denote an oxygen atom (O) or an N—R group where each R radical independently denotes a hydrogen atom (H) or an R¹ radical;
Y is a divalent group;
R¹, R¹¹, R⁴, R¹⁴, R⁵ and R¹⁵ independently denote a substituted or unsubstituted hydrocarbyl group;
R³ and R¹³ independently denote a divalent group;
w and ww are independently integers ranging from 0 to 1000;
x and xx are, independently, integers ranging from 1 to 100;
y and yy are, independently, integers ranging from 0 to 1000;
preferably, w and y are not simultaneously equal to 0 and ww and yy are not simultaneously equal to 0.

The term “substituted” means one or more hydrogen atoms of the hydrocarbyl group are replaced by an atom other than hydrogen (i.e. halogen) or else that one or more carbon atoms are replaced by an atom other than carbon such as a heteroatom such as oxygen, sulfur or nitrogen.

In formula (I), the lower and upper portions are identical or different siloxanes. According to certain specific embodiments, each siloxane chain comprises siloxane bonds (Si—O—Si) in their respective backbone. Each siloxane chain may include siloxane bonds separated by one or more divalent groups, for example a —CH₂— linking group.

Other examples of divalent groups may include polyether groups: for example —CH₂CH₂O— (i.e. ethylene oxide group), —CH(CH₃)CH₂O— (i.e. propylene oxide), etc. linking groups. Combinations of different divalent groups may be present in their respective backbone. Each of the divalent groups may be singular or repeated, i.e.: 2 times, 5 times, 10 times, or even >10 times, etc. According to certain embodiments, the siloxanes do not contain any polyether groups.

In various embodiments, one or both siloxanes may comprise at least one [SiR¹R²—O-] unit (“D” or R*₂SiO_{2/2} units). Typically, each siloxane has a repetition of D units, which generally constitutes the linear portions of the siloxanes. The siloxanes also typically have terminal R*₃SiO_{1/2} units (“M” units).

In certain specific embodiments, one of the two siloxanes or both siloxanes may optionally be branched, partially branched and/or include a resin portion having a three-dimensional structure network. In such cases, the corresponding siloxane(s) may additionally comprise R*SiO_{3/2} units (“T” units) and/or SiO_{4/2} units (“Q” units). The branched or resinous nature of the siloxane(s) can be attributed to the presence of T and/or Q units. The branched nature of a siloxane may be attributed to side groups of one or more D units. According to particular embodiments, one or both of the siloxanes may be devoid of T units and/or of units. The two siloxanes may be identical or different: i.e. one is linear and the other is branched, or else both siloxanes are linear.

According to certain specific embodiments, the R¹ radicals independently denote an alkyl, aryl, alkenyl, alkaryl, or aralkyl radical. More particularly, the R¹ radicals denote an alkyl radical, preferably having from 1 to 20, from 1 to 15, from 1 to 10, from 1 to 6, from 1 to 4 or 1 or 2 carbon atoms, such as, for example, methyl, ethyl, propyl, butyl or pentyl.

More particularly, all the R¹ radicals denote a methyl group (i.e. —CH₃).

Preferably, the R³ radical is a hydrocarbylene, heterohydrocarbylene, or organoheterylene group. In particular, R³ is a (CH₂)ₙ group where n is an integer ranging from 1 to 30, from 1 to 25, from 1 to 20, from 1 to 15, from 1 to 10, from 1 to 5, or from 1 to 3, and more particularly equal to 3.

According to various specific embodiments, each of the R⁴ and R⁵ radicals may independently denote a substituted or unsubstituted hydrocarbyl group, notably chosen from those defined above for the R¹ radicals.

In some embodiments, each R⁴ radical independently denotes an alkyl or aryl (i.e. phenyl) group or an (R⁶O)ₘ group. When R⁴ is an (R⁶O)ₘ group, R⁶ is preferably an alkyl or aryl (i.e.: phenyl) radical and m is an integer ranging from 1 to 50, from 1 to 25, from 1 to 10, or from 1 to 5, or is 1. The (R⁶O)ₘ group may be a polyether group (i.e.: with ethylene oxide and/or propylene oxide units). According to particular embodiments, an R⁴ radical may be a divalent group of a silicone side chain of the siloxane.

According to a specific embodiment, R⁴ may denote an anhydride group of formula (1)

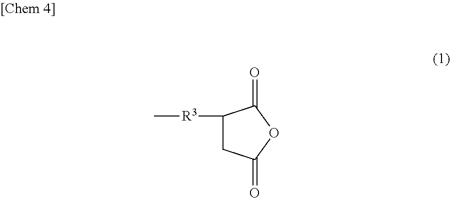

in which R³ has the same definition indicated previously.

Preferably, the R⁴ radicals denote an alkyl radical or a polyether group.

Preferably, each R⁵ radical is an R¹ radical. For example, each R⁵ and R¹ radical is an alkyl group: i.e. methyl.

Preferably, R¹¹ is identical to or different from R¹, R¹⁴ is identical to or different from R⁴, and R¹⁵ is identical to or different from R⁵. Preferentially, the R⁵ radicals denote R¹ and/or the R¹⁵ radicals denote R¹¹.

Preferably, w is an integer ranging from 0 to 1000, from 0 to 950, from 0 to 750, from 0 to 500, from 0 to 400, from 1 to 350, from 1 to 300, from 25 to 250, from 50 to 200, from 50 to 150, from 75 to 125, from 90 to 110, from 90 to 100, from 90 to 95, and more particularly is equal to 93.

Preferably, x is an integer from 1 to 100, from 1 to 75, from 1 to 50, from 1 to 25, from 1 to 20, from 1 to 10, or from 1 to 5, and more particularly equal to 3.

Preferably, y is an integer ranging from 0 to 1000, from 0 to 950, from 0 to 750, from 0 to 500, from 0 to 400, from 1 to 350, from 1 to 300, from 1 to 250, from 1 to 200, from 1 to 150, from 1 to 100, from 1 to 75, from 1 to 50, from 1 to 25, from 1 to 20, from 1 to 15, from 1 to 10, or from 1 to 5.

According to various embodiments, w and y are not simultaneously equal to 0. In certain embodiments, the sum w+x+y varies from 25 to 1500, from 25 to 1000, from 25 to 900, from 25 to 800, from 25 to 700, from 25 to 600, from 25 to 500, from 25 to 400, from 25 to 300, from 50 to 200, from 75 to 150, from 85 to 125 or from 90 to 110.

In certain embodiments, x is at least equal to 1, at least equal to 10, at least equal to 25, at least equal to 50, at least equal to 75 or at least equal to 5.

According to specific embodiments, ww may be identical to or different from w, xx may be identical to or different from x and yy may be identical to or different from y. The units of formula (I), notably those represented between brackets with the subscripts w, ww, x, xx, y or yy, may be present in any order, randomly or in block form.

According to certain particular embodiments, the R¹¹ radicals, which are identical or different, denote a linear, branched or cyclic C₁-C₂₀, C₁-C₁₅, C₁-C₁₀, C₁-C₆, C₁-C₄ or C₁-C₂ alkyl radical, such as methyl, ethyl, propyl, butyl, pentyl, etc. groups. More preferentially, the R¹¹ radicals are a methyl group.

According to certain specific embodiments, the R¹⁴ radicals, which are identical or different, denote an alkyl or aryl (i.e. phenyl) radical or (R¹⁶O)ₘₘ. When R¹⁴ is an (R¹⁶O)ₘₘ group, R¹⁶ denotes an alkyl or aryl (i.e. phenyl) group and mm is an integer ranging from 1 to 50, 1 to 25, 1 to 10, 1 to 5, or 1. The group (R¹⁶O)ₘₘ may be a polyether group (i.e.: with ethylene oxide and/or propylene oxide units).

According to specific embodiments, the R¹⁴ radicals denote, independently, an alkyl radical having from 2 to 20, 2 to 15, 2 to 10, 2 to 5, or 2 carbon atoms.

According to other specific embodiments, R¹⁴ may be a divalent linking group of a silicone side chain of the siloxane.

According to a specific embodiment, R¹⁴ may denote an anhydride group of formula (1)

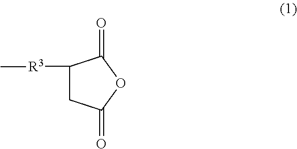

in which R³ has the same definition indicated previously.

Preferably, the R¹⁴ radicals denote an alkyl radical or a polyether group.

Preferably, ww is an integer ranging from 0 to 1000, from 0 to 950, from 0 to 750, from 0 to 500, from 0 to 400, from 1 to 350, from 1 to 300, from 25 to 250, from 50 to 200, from 50 to 150, from 75 to 125, from 90 to 110, from 90 to 100, from 90 to 95, and more particularly 93.

Preferably, xx is an integer ranging from 1 to 100, from 1 to 75, from 1 to 50, from 1 to 25, from 1 to 20, from 1 to 10, or from 1 to 5, and more particularly 3.

Preferably, yy is an integer ranging from 0 to 1000, from 0 to 950, from 0 to 750, from 0 to 500, from 0 to 400, from 1 to 350, from 1 to 300, from 1 to 250, from 1 to 200, from 1 to 150, from 1 to 100, from 1 to 75, from 1 to 50, from 1 to 25, from 1 to 20, from 1 to 15, from 1 to 10 or from 1 to 5.

In various embodiments, ww and yy are not simultaneously equal to 0. According to certain specific embodiments, the sum ww+xx+yy varies from 25 to 1500, from 25 to 1000, from 25 to 900, from 25 to 800, from 25 to 700, from 25 to 600, from 25 to 500, from 25 to 400, from 25 to 300, from 50 to 200, from 75 to 150, from 85 to 125, or from 90 to 110.

According to certain specific embodiments, xx is at least equal to 1, at least equal to 10, at least equal to 25, at least equal to 50, at least equal to 75 or at least equal to 85.

The portion of the middle X of formula (I) corresponds to the radical X corresponds to formula (i) or formula (i′) below:

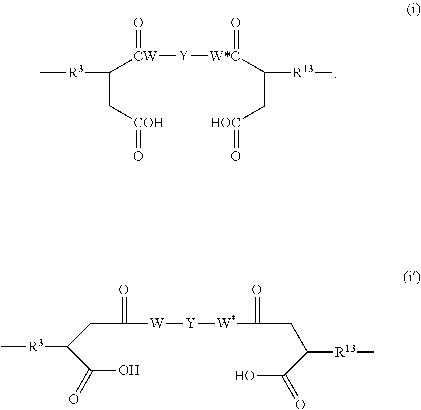

in which
each group W and W* independently denotes an oxygen atom (O) or an N—R group, with R denoting a hydrogen atom (H) or a radical R¹; preferably, R is H;
R¹³ is a divalent group, preferably is a hydrocarbylene, a heterohydrocarbylene or an organoheterylene; in particular, R¹³ denotes a radical (CH₂)ₙₙ where nn is an integer from 1 to 30, from 1 to 20, from 1 to 15, from 1 to 10, from 1 to 5, from 1 to 3, and more particularly is 3; R¹³ can be identical to or different from R³;
each Y radical is a divalent group, notably denotes a hydrocarbylene, a heterohydrocarbylene or an organoheterylene radical; in particular, Y is a hydrocarbylene group having from 1 to 50, from 1 to 40, from 1 to 30, from 1 to 20, from 1 to 10, from 1 to 5, 1 or 2 carbon atoms or may denote a group of formula (ii) below:

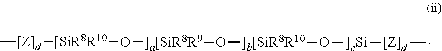

in which
each R⁸, R⁹ denote, independently, a substituted or unsubstituted hydrocarbyl group, notably chosen from an alkyl group (i.e. methyl), an aryl group (i.e. phenyl), a polyether group (i.e. with ethylene oxide and/or propylene oxide units);
each R¹⁰ radical denotes, independently, a substituted or unsubstituted hydrocarbyl group such as the radicals defined previously for R¹ and R⁴; in particular, R¹⁰ may be an alkyl group having from 1 to 20 carbon atoms or a polyether group or a divalent linking group of a silicone side chain of the siloxane;
each radical Z independently comprises at least one hydrocarbylene, heterohydrocarbylene or organoheterylene group, and in particular is a hydrocarbylene group having from 1 to 20, from 1 to 10, from 1 to 5, or 1 carbon atom(s); notably, Z may include siloxane bonds separated by one or more divalent groups, for example a —CH₂— linking group; other examples of divalent groups can include polyether groups: for example, —CH₂CH₂O— (i.e. ethylene oxide group), —CH(CH₃)CH₂O— (i.e. propylene oxide) linking groups;
a is an integer ranging from 0 to 1000, from 0 to 950, from 0 to 750, from 0 to 500, from 0 to 400, from 0 to 300, from 0 to 200, from 0 to 100, from 0 to 75, from 0 to 50, from 0 to 25, from 0 to 20,
b is an integer ranging from 1 to 1000, from 1 to 950, from 1 to 750, from 1 to 500, from 1 to 400, from 1 to 300 or from 1 to 200;
c is an integer ranging from 0 to 1000, from 0 to 950, from 0 to 750, from 0 to 500, from 0 to 400, from 0 to 300, from 0 to 200, from 0 to 100;
each d is equal to 0 or 1. In particular, at least one of the d is equal to 1 or both are equal to 1;
   the units of formula (ii) represented between brackets with the subscripts a, b or c being present in any order, randomly or in block form.

According to a specific form, the siloxane of formula (ii) may be a resin of formula R*ₛSiO_{(4-s)/2}. Preferably, a silicone resin has, along its chain, T and/or Q units with M units and optionally D units. The R* radicals may be chosen independently from a substituted hydrocarbonyl group (i.e.: hydroxyl, amine functions) or an unsubstituted hydrocarbonyl group and s varies from 0 to 3. The R* groups that can be used may be those described previously for the R⁸, R⁹, and R¹⁰ radicals. Various combinations of such groups may be present. According to these particular cases, the silicone resin comprises a combination of M, D, T and/or Q units. It may in particular be a resin of the MDT type, a resin of the MT type, a resin of the MDQ type, a resin of the MQ type or a resin of the MDTQ type. Each of the M, D, and T units can contain different R* groups. The resin can have various molecular weights such as a number average molecular weight ranging from 800 to 500 000.

According to a specific form of the invention, the silicone elastomers containing carboxylic acid functions have a carboxyl equivalent ranging from 100 to 50 000, from 500 to 10 000, or 500 to 5000.

The silicone elastomers containing carboxylic acid functions of formula (I) may be obtained according to one of the synthesis processes described in document WO2015066161 (US20160200876).

According to a first variant of the synthesis process, certain silicone elastomers containing carboxylic acid functions may be obtained by reaction 1) of a first siloxane bearing pendent anhydride groups, 2) of a second siloxane bearing pendent anhydride groups and 3) of an organic polyol. According to a specific embodiment, the 3 preceding reactants can be reacted with one or more siloxanes different from the first two.

According to a second variant of the synthesis process, certain silicone elastomers containing carboxylic acid functions may be obtained by reaction 1) of a first siloxane bearing pendent anhydride groups, 2) of a second siloxane bearing pendent anhydride groups and 3) of an organic polyamine. According to a specific embodiment, the 3 preceding reactants can be reacted with one or more additional siloxanes different from the first two.

According to a third variant of the synthesis process, certain silicone elastomers containing carboxylic acid functions can be obtained by reaction 1) of a first siloxane bearing pendent anhydride groups, 2) of a second siloxane bearing pendent anhydride groups and 3) of a third siloxane having at least two hydroxyl groups. According to a specific embodiment, the 3 preceding reactants can be reacted with one or more additional siloxanes different from the first two.

According to a fourth variant of the synthesis process, certain silicone elastomers containing carboxylic acid functions may be obtained by reaction 1) of a first siloxane bearing pendent anhydride groups, 2) of a second siloxane bearing pendent anhydride groups and 3) of a third siloxane having pendent amine groups and/or terminal amine groups. According to a specific embodiment, the 3 preceding reactants can be reacted with one or more additional siloxanes different from the first two.

According to a fifth variant of the synthesis process, certain silicone elastomers containing carboxylic acid functions may be obtained by reaction 1) of a first siloxane bearing pendent hydroxyl groups, 2) of a second siloxane bearing pendent hydroxyl groups and 3) of a third siloxane having at least two terminal anhydride groups. According to a specific embodiment, the 3 preceding reactants can be reacted with one or more additional siloxanes different from the first two.

According to a sixth variant of the synthesis process, certain silicone elastomers containing carboxylic acid functions may be obtained by reaction 1) of a first siloxane bearing pendent amine groups, 2) of a second siloxane bearing pendent amine groups and 3) of a third siloxane having at least two terminal anhydride groups. According to a specific embodiment, the 3 preceding reactants can be reacted with one or more additional siloxanes different from the first two.

According to a seventh variant of the synthesis process, certain silicone elastomers containing carboxylic acid functions may be obtained by reaction 1) of a first organic alcohol, 2) of a second organic alcohol and 3) of a siloxane having terminal anhydride groups. According to a specific embodiment, the 3 preceding reactants can be reacted with one or more additional organic alcohols different from the first two.

Among the silicone elastomers containing carboxylic acid functions of formula (I), mention may be made of the following compounds 1 to 12 as described respectively in Examples 4, 5 and 7 to 16 of application WO2015066161 and compound 13 as described in application US20160200876 (Example 15) and the examples of the patent:

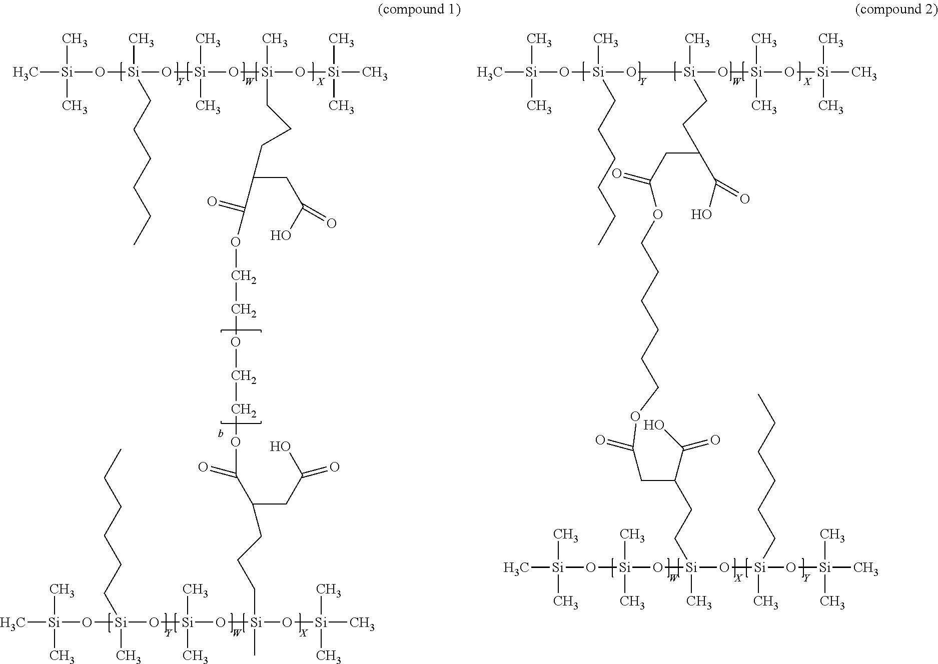

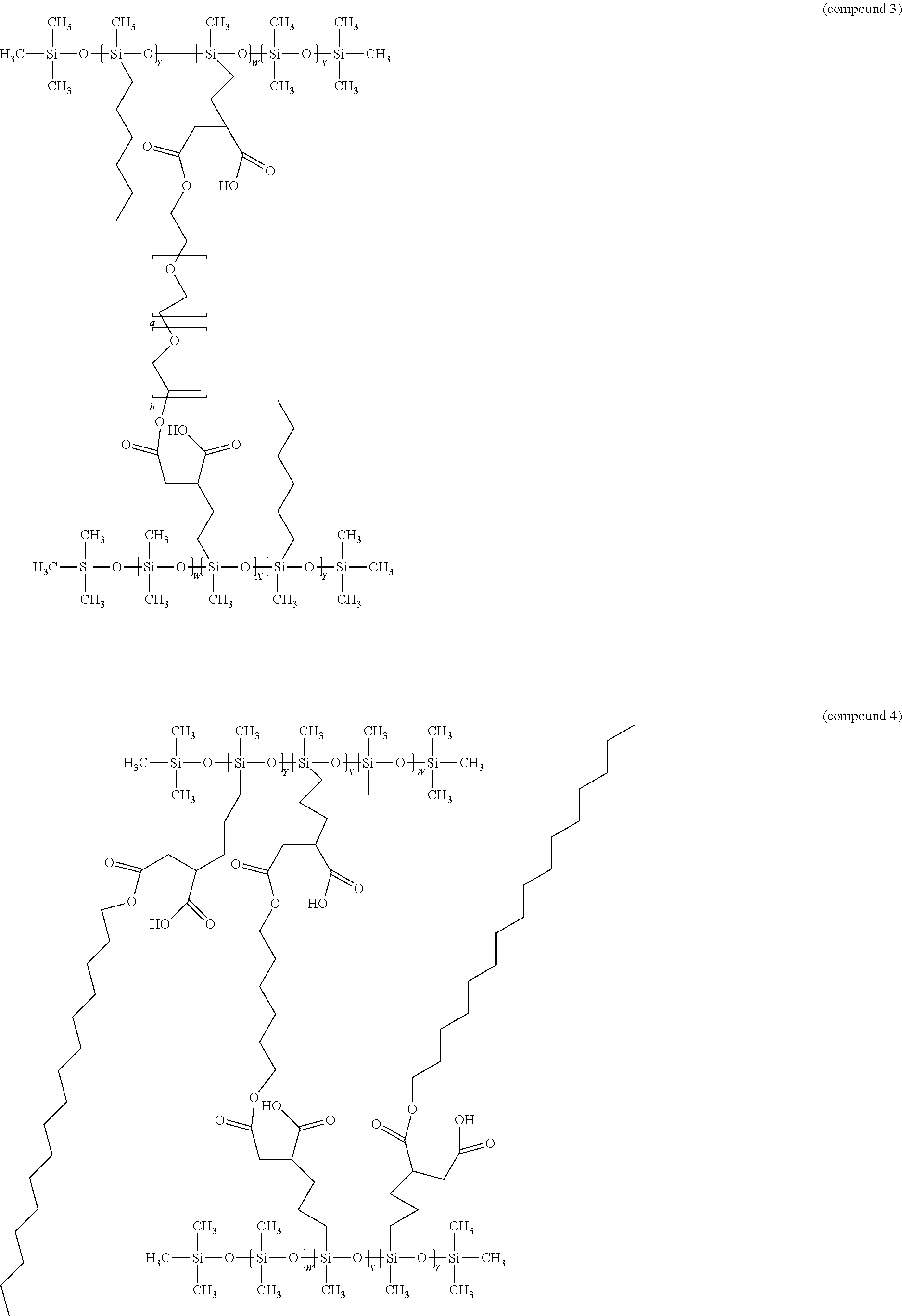

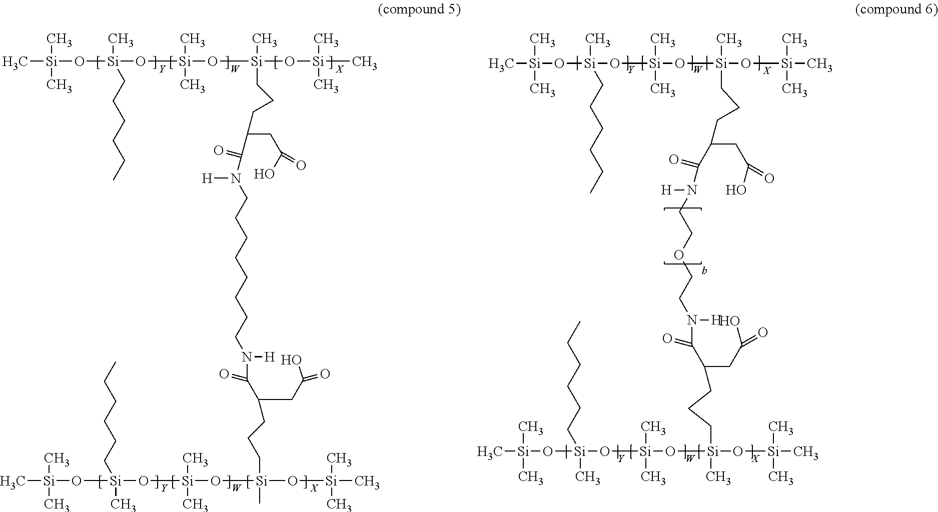

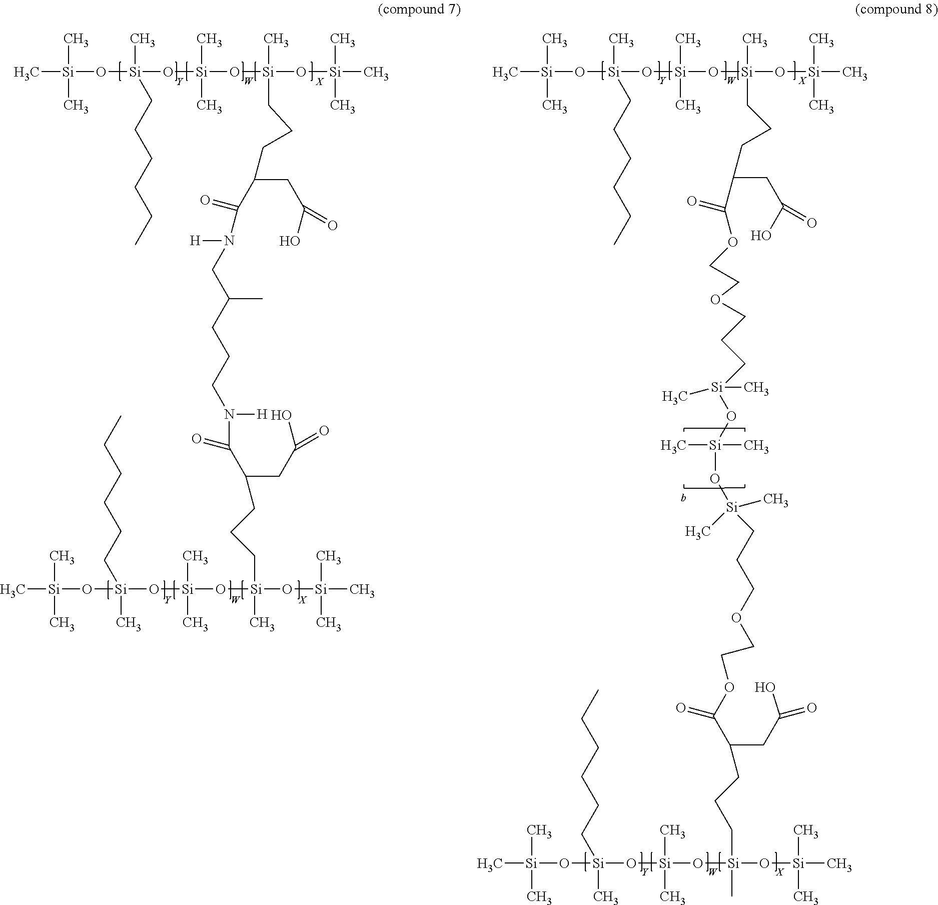

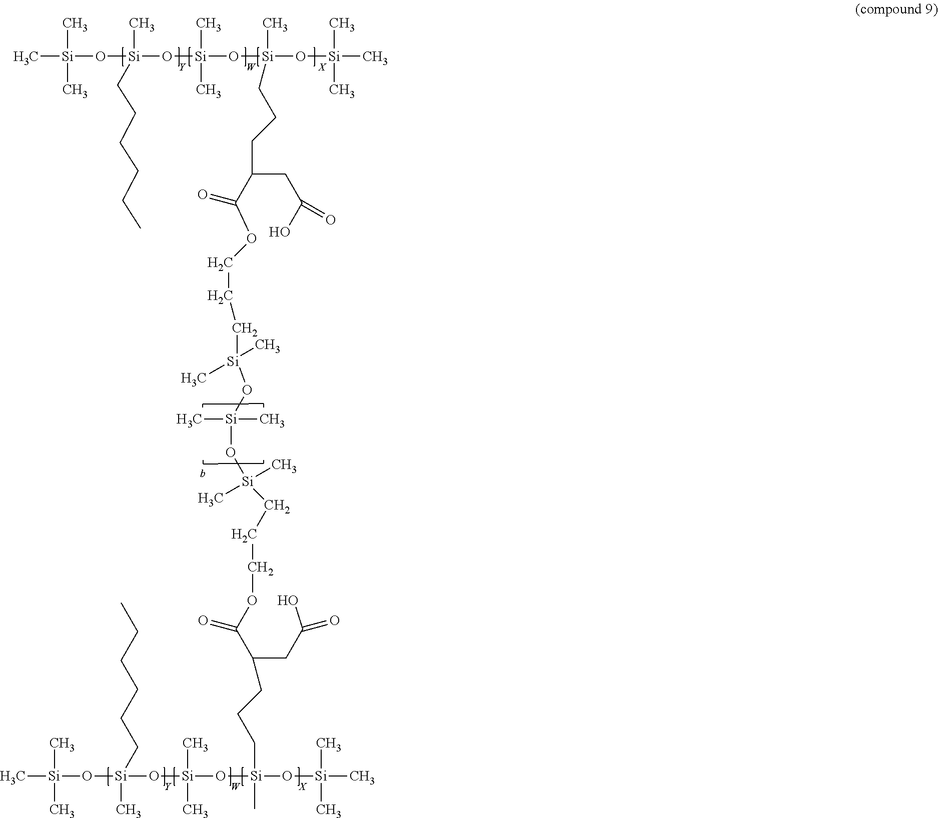

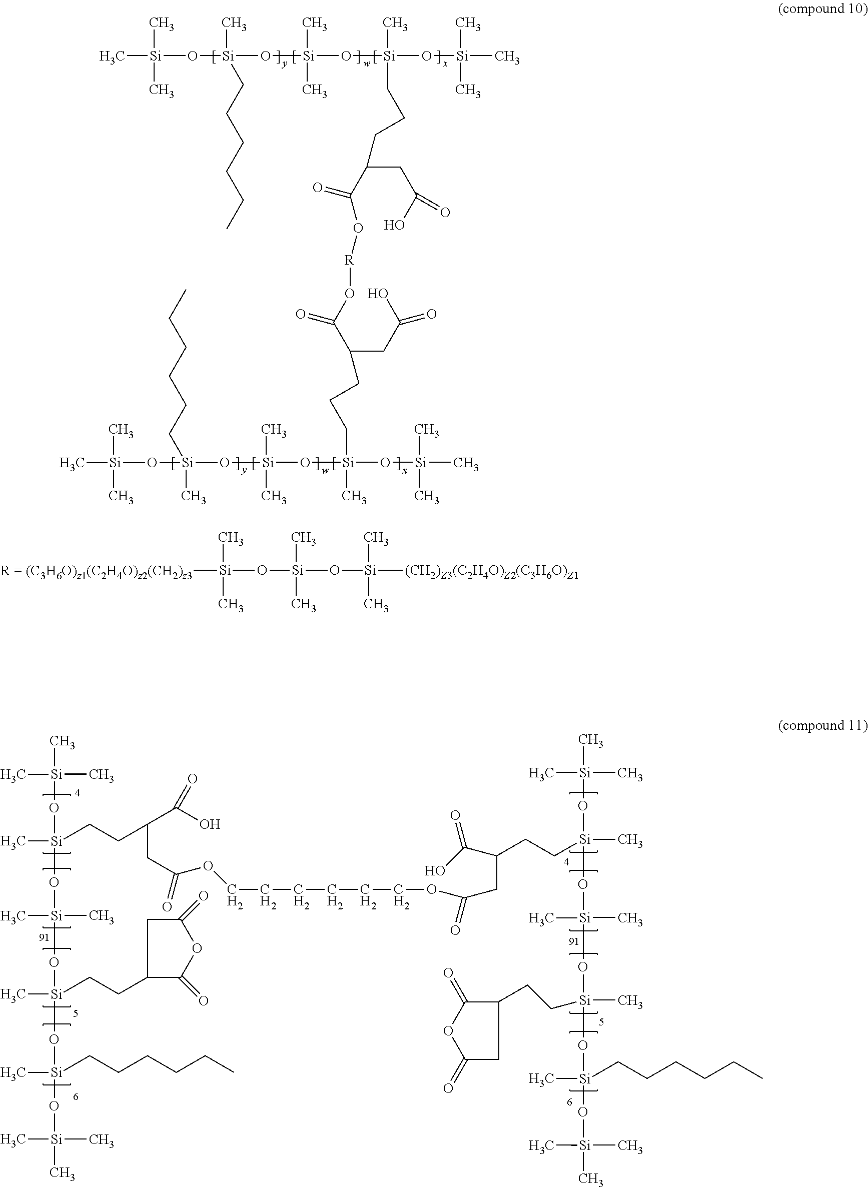

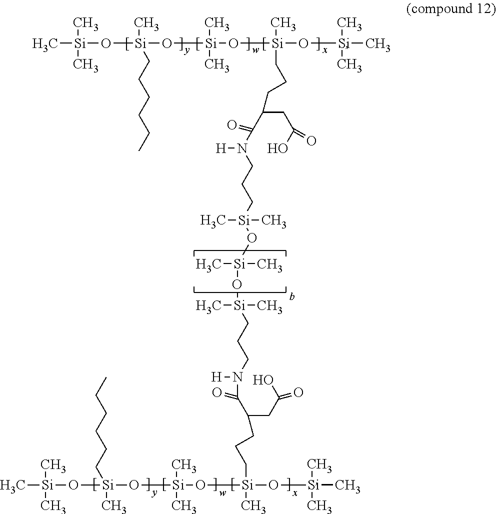

According to one particular form, the composition of the invention contains at least compound 10 or compound 11 as defined above. Compound 10 is described in particular in document US20160200876 (compound of Example 15) and the examples of patent U.S. Pat. No. 10,918,587B2.

Among the silicone elastomers containing carboxylic acid functions that can be used in the compositions of the invention, mention may also be made of the silicone elastomer with the INCI name: Hexyl/Succinyl Dimethicone Crosspolymer.

According to a specific form, the silicone elastomers containing carboxylic acid functions are in solid form. They may notably be chosen from compounds 2 and 3 as defined previously.

According to another particular form, the silicone elastomers containing carboxylic acid functions are in dispersion in at least one oil H₂ and are in gel form.

The oil H₂ may be chosen from volatile oils and/or non-volatile oils.

The term “non-volatile oil” is understood to mean an oil which remains on the keratin material at ambient temperature (25° C.) and atmospheric pressure (760 mmHg) for at least several hours and which has in particular a vapour pressure of less than 10⁻³ mmHg (0.13 Pa).

The oil(s) H₂ may be chosen from hydrocarbon oils and silicone oils, and mixtures thereof.

For the purposes of the present invention, the term “silicone oil” is understood to mean an oil comprising at least one Si—O group, and more particularly an organopolysiloxane.

Mention may be made, among the volatile silicone oils H₂ which can be used in the compositions, for example, of volatile linear or cyclic silicone oils, in particular those having a viscosity of less than or equal to 8 centistokes (8×10⁻⁶ mm²/s) and having in particular from 2 to 7 silicon atoms, these silicones optionally comprising alkyl or alkoxy groups having from 1 to 10 carbon atoms.

As linear volatile silicone oil H₂ which can be used in the invention, mention may be made of octamethyltrisiloxane, decamethyltetrasiloxane, dodecamethylpentasiloxane, and mixtures thereof.

As cyclic volatile silicone oil H₂ which can be used in the invention, mention may be made of cyclomethicones such as octamethylcyclotetrasiloxane, decamethylcyclopentasiloxane, dodecamethylcyclohexasiloxane, and mixtures thereof.

Mention may be made, as examples of linear non-volatile silicone oil H₂, of polydimethylsiloxanes (also known as dimethicones); alkyl dimethicones; vinylmethylmethicones and polydimethylsiloxanes modified by aliphatic groups and/or functional groups, such as hydroxyl, thiol, carboxylic acid and/or amine groups.

Mention may be made, as examples of non-volatile linear hydrocarbon oils, of:
synthetic esters, such as oils of the formula R¹COOR₂, in which R₁ represents a residue of a linear or branched fatty acid comprising from 1 to 40 carbon atoms, and R₂ represents a hydrocarbon chain, which is in particular branched, comprising from 1 to 40 carbon atoms provided that the sum of the number of carbon atoms in the R1 and R2 chains is greater than or equal to 10; the esters may be chosen, in particular, from esters of an alcohol and of a fatty acid, such as, for example, ketostearyl octanoate, isopropyl alcohol esters, such as isopropyl myristate, isopropyl palmitate, ethyl palmitate, 2-ethylhexyl palmitate, isopropyl stearate or isostearate, isostearyl isostearate, octyl stearate, hydroxylated esters such as isostearyl lactate, octyl hydroxystearate, diisopropyl adipate, heptanoates, and in particular isostearyl heptanoate, octanoates, decanoates or ricinoleates of alcohols or of polyalcohols, such as propylene glycol dioctanoate, cetyl octanoate, tridecyl octanoate, 2-ethylhexyl palmitate and 4-diheptanoate, alkyl benzoate, polyethylene glycol diheptanoate, propylene glycol diethyl 2-hexanoate, and mixtures thereof, C₁₂-C₁₅ alkyl benzoates, hexyl laurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, hydroxylated esters, for instance isostearyl lactate and diisostearyl malate; and more particularly isodecyl neopentanoate;
dialkyl carbonates, the two alkyl chains possibly being identical or different, such as the dicaprylyl carbonate sold under the name Cetiol CC® by Cognis;
synthetic ethers containing from 10 to 40 carbon atoms, such as dicaprylyl ether.

According to a preferred form of the invention, the oil(s) H₂ may be chosen from the volatile hydrocarbon oils as described above for oils H₁. The oil(s) H₂ may be identical to or different from the oil(s) H₁ present in the oily phase of the composition.

According to a particular form of the invention, the oil(s) H₂ present in the oily dispersion of silicone elastomer containing carboxylic acid functions is (are) identical to the oil(s) H₁ present in the oily phase of the composition.

According to a more particularly preferred form of the invention, the oil H₂ present in the oily dispersion of silicone elastomer containing carboxylic acid functions and the oil H₁ present in the oily phase of the composition denote a C₈-C₁₆ isoalkane of petroleum origin, and more particularly isododecane.

According to a preferred form, the oily dispersion of silicone elastomer containing carboxylic acid functions comprises from 10% to 40% by weight of silicone elastomer active material, and more preferentially from 20% to 35% by weight relative to the total weight of the oily dispersion.

According to a preferred form, the composition of the invention comprises at least one silicone elastomer containing carboxylic acid functions in dispersion in isododecane and in gel form. In particular, said silicone elastomer is chosen from compounds 1 and 4 to 11 as defined above.

According to a particularly preferred form, the composition of the invention comprises compound 10 or compound 11 in dispersion in isododecane and in gel form, and more particularly in an active material content equal to 30% by weight relative to the total weight of the dispersion.

According to another particularly preferred form, the composition of the invention comprises a silicone elastomer containing carboxylic acid functions in dispersion in isododecane and in gel form with the INCI name: Isododecane (and) Hexyl/Succinyl Dimethicone Crosspolymer and more particularly in an active material content equal to 30% by weight relative to the total weight of the dispersion, such as the commercial product Dowsil EL-7314 Silicone Elastomer Blend® sold by Dow Corning, comprising 30% by weight of active material of said silicone elastomer.

According to the present invention, the weight ratio of the total amount of volatile hydrocarbon oil(s) relative to the total weight of the composition to the amount of silicone elastomer containing carboxylic acid functions relative to the total weight of the composition ranges from 3.8 to 12.0, and more preferentially from 4.2 to 10.

When the composition of the invention comprises an oily dispersion of silicone elastomer containing carboxylic acid functions in dispersion in at least one volatile hydrocarbon oil H₂, the weight ratio of the total amount of volatile hydrocarbon oil(s) H₁ and H₂ relative to the total weight of the composition to the amount of silicone elastomer containing carboxylic acid functions relative to the total weight of the composition ranges from 3.8 to 12.0 and more preferentially from 4.2 to 10.

Silicone Resins and/or Copolymers of a Silicone Resin

According to a particular form, the composition of the invention may additionally comprise at least one silicone resin and/or at least one copolymer of a silicone resin and of a silicone of dimethiconol type.

### (A) Silicone Resin

The nomenclature of silicone resins (also known as siloxane resins) is known under the name “MDTQ”, the resin being described as a function of the various siloxane monomer units it comprises, each of the letters “MDTQ” characterizing a type of unit.

The letter “M” represents the monofunctional unit of formula R1R2R3SiO_{1/2}, the silicon atom being bonded to only one oxygen atom in the polymer comprising this unit.

The letter “D” signifies a difunctional unit R1R2SiO_{2/2} in which the silicon atom is bonded to two oxygen atoms.

The letter “T” represents a trifunctional unit of formula R1SiO_{3/2}.

Such resins are described, for example, in the Encyclopedia of Polymer Science and Engineering, vol. 15, John Wiley & Sons, New York, (1989), pp. 265-270, and U.S. Pat. Nos. 2,676,182, 3,627,851, 3,772,247, 5,248,739 or U.S. Pat. Nos. 5,082,706, 5,319,040, 5,302,685 and 4,935,484.

In the units M, D and T defined previously, R, namely R1, R2 and R3, represents a hydrocarbon radical (especially alkyl) containing from 1 to 10 carbon atoms, a phenyl group, a phenylalkyl group or a hydroxyl group.

Finally, the letter “Q” signifies a tetrafunctional unit SiO_{4/2} in which the silicon atom is bonded to four oxygen atoms, which are themselves bonded to the remainder of the polymer.

Various silicone resins with different properties can be obtained from these different units, the properties of these polymers varying as a function of the type of monomer (or units), of the nature and number of the R radical, of the length of the polymer chain, of the degree of branching and of the size of the pendent chains.

Use may be made, as silicone resins which can be used in the compositions according to the invention, for example, of silicone resins of MQ type, of T type or Q of MQT type.

### MQ Resins:

As examples of silicone resins of MQ type, mention may be made of the alkyl siloxysilicates of formula [(R1)₃SiO_{1/2}]x(SiO_{4/2})y (MQ units) in which x and y are integers ranging from 50 to 80, and such that the group R1 represents a radical as defined previously, and is preferably an alkyl group containing from 1 to 8 carbon atoms, or a hydroxyl group, preferably a methyl group.

As examples of MQ silicone resins of trimethylsiloxysilicate type, mention may be made of those sold under the reference SR1000® by the company General Electric, under the reference TMS 803@ by the company Wacker, or under the name KF-7312J® by the company Shin-Etsu or DC 749® or DC 593® by the company Dow Corning.

As silicone resins comprising MQ siloxysilicate units, mention may also be made of phenylalkylsiloxysilicate resins, such as phenylpropyldimethylsiloxysilicate (Silshine 151® sold by the company General Electric). The preparation of such resins is notably described in patent U.S. Pat. No. 5,817,302.

### T Resins:

Examples of silicone resins of type T that may be mentioned include the polysilsesquioxanes of formula (RSiO_{3/2})x (T units) in which x is greater than 100 and such that the group R is an alkyl group containing from 1 to 10 carbon atoms, said polysilsesquioxanes also possibly comprising Si—OH end groups.

Polymethylsilsesquioxane resins that may preferably be used are those in which R represents a methyl group, for instance those sold:
by the company Wacker under the reference Resin MK®, such as Belsil PMS MK®: polymer comprising CH₃SiO_{3/2} repeating units (T units), which may also comprise up to 1% by weight of (CH₃)₂SiO_{2/2} units (D units) and having an average molecular weight of about 10 000 g/mol, or
by the company Shin-Etsu under the reference KR-220L®, which are composed of T units of formula CH₃SiO_{3/2} and contain Si—OH (silanol) end groups, under the reference KR-242AR, which comprise 98% of T units and 2% of dimethyl D units and contain Si—OH end groups, or else under the reference KR-251®, comprising 88% of T units and 12% of dimethyl D units and contain Si—OH end groups.

### MQT Resins:

Resins comprising MQT units that are notably known are those mentioned in U.S. Pat. No. 5,110,890.

A preferred form of resins of MQT type are MQT-propyl (also known as MQTPr) resins. Such resins that may be used in the compositions according to the invention are notably the resins described and prepared in patent application WO 2005/075542, the content of which is incorporated herein by reference.

The MQ-T-propyl resin preferably comprises the units:

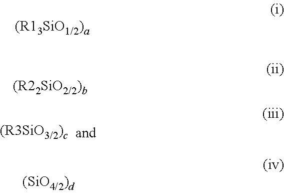

with
R1, R2 and R3 independently representing a hydrocarbon radical (notably alkyl) containing from 1 to 10 carbon atoms, a phenyl group, a phenylalkyl group or a hydroxyl group and preferably an alkyl radical containing from 1 to 8 carbon atoms or a phenyl group,
a, b, c and d being mole fractions,
a being between 0.05 and 0.5,
b being between 0 and 0.3,
c being greater than zero,
d being between 0.05 and 0.6,
a+b+c+d=1,
provided that more than 40 mol % of the R³ groups of the siloxane resin are propyl groups.

Preferably, the siloxane resin comprises the units:

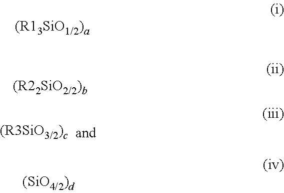

with R1 and R3 independently representing an alkyl group containing from 1 to 8 carbon atoms, R1 preferably being a methyl group and R3 preferably being a propyl group,
a being between 0.05 and 0.5 and preferably between 0.15 and 0.4,
c being greater than zero, preferably between 0.15 and 0.4,
d being between 0.05 and 0.6, preferably between 0.2 and 0.6 or alternatively between 0.2 and 0.55,
a+b+c+d=1, and a, b, c and d being mole fractions,
provided that more than 40 mol % of the R3 groups of the siloxane resin are propyl groups.

The siloxane resins which can be used according to the invention can be obtained by a process comprising the reaction of:
A) an MQ resin comprising at least 80 mol % of units (R1₃SiO_{1/2})ₐ and (SiO_{4/2})_{d} with R1 representing an alkyl group having from 1 to 8 carbon atoms, an aryl group,
a carbinol group or an amino group,
a and d being greater than zero, the ratio a/d being between 0.5 and 1.5; and of
B) a T-propyl resin comprising at least 80 mol % of units (R3SiO_{3/2})_{c},
with R3 representing an alkyl group having from 1 to 8 carbon atoms, an aryl group, a carbinol group or an amino group,
c being greater than zero,
provided that at least 40 mol % of the R3 groups are propyl groups,
where the weight ratio A/B is between 95:5 and 15:85 and preferably the weight ratio A/B is 30:70.

Advantageously, the weight ratio A/B is between 95:5 and 15:85. Preferably, the ratio A/B is less than or equal to 70:30. These preferred ratios have proven to afford comfortable deposits.

Preferably, the composition according to the invention comprises, as silicone resin, at least one resin of MQ type, more particularly of trimethylsiloxysilicate type, such as those sold under the reference SR1000® by the company General Electric, under the reference TMS 803® by the company Wacker, or under the name KF-7312J® by the company Shin-Etsu or DC 749® or DC 593® by the company Dow Corning.

According to a particular embodiment of the invention, the silicone resin is present in the composition in a resin solids content ranging from 8% to 40% by weight relative to the total weight of the composition, preferably ranging from 10% to 35% by weight and more preferentially from 15% to 30% by weight relative to the weight of composition (A).

### B) Copolymer Based on Silicone Resin and Silicone of Dimethiconol Type.

Such copolymers are described for example in “Silicone Pressure Sensitive Adhesives”, Sobieski and Tangney, Handbook of Pressure Sensitive Adhesive Technology (D. Satas Ed.), Van Nostrand Reinhold, New York.

In the copolymer, the silicone resin is present at a content of between 45% and 75% (relative to the total weight of silicone) and the silicone of dimethiconol type is present at a content of between 25% and 55%, with the sum of the percentages of silicone resin and silicone of dimethiconol type being equal to 100. Preferably, the silicone resin is present at a content of between 55% and 65% (relative to the total weight of silicone) and the silicone of dimethiconol type is present at a content of between 35% and 45%, with the sum of the percentages of silicone resin and silicone of dimethiconol type being equal to 100.

Preferably, the silicone resin according to the invention is the condensation product of SiO₂ groups and R₃(SiO)_{1/2} (triorganosilyl) groups for which each R group is independently selected from methyl, ethyl, propyl or vinyl radicals and for which the ratio between the SiO₂ functions and the R₃(SiO)_{1/2} functions of the silicone resin ranges from 0.6 to 0.9.

Triorganosilyl groups that can be used to form the silicone resin may be trimethylsilyl, triethylsilyl, methylmethylpropylsilyl or dimethylvinylsilyl units and mixtures thereof. The trimethylsilyl group is the preferred group within the context of the invention.

Preferably, the silicone of dimethiconol type according to the invention is a diorganopolysiloxane with terminal OH functions which has a viscosity of between 100 and 100 000 cSt at 25° C. and for which the substituents of the diorganopolysiloxane are chosen independently from methyl, ethyl, propyl and vinyl radicals. The diorganopolysiloxanes are preferably linear polymers. Examples of diorganopolysiloxanes may be, without limitation, a polydimethylsiloxane, an ethylmethylpolysiloxane, a copolymer of dimethylsiloxane and methylvinylsiloxane, and mixtures of such polymers or copolymers having OH ends. The preferred diorganopolysiloxane is a polydimethylsiloxane.

Examples of synthesis of such a copolymer are for example described in patent U.S. Pat. No. 5,162,410 or in patent CA 711756.

The copolymers according to the present invention may thus be prepared by heating the following mixture:
from 45% to 75% by mass of silicone resin which is the product of condensation of units SiO₂ and R₃(SiO)_{1/2} for which each group R is independently selected from methyl, ethyl, propyl and vinyl radicals and for which the ratio between the SiO₂ functions and the R₃(SiO)_{1/2} functions in the silicone resin ranges from 0.6 to 0.9;
from 25% to 55% by mass of diorganopolysiloxane with terminal OH functions which has a viscosity of between 100 and 100 000 cSt at 25° C. and for which the substituents of the diorganopolysiloxane are independently chosen from methyl, ethyl, propyl and vinyl radicals;
from 0.001% to 5% of a suitable catalyst, which is preferably an organic aliphatic amine compound preferably chosen from primary amines, secondary amines, tertiary amines, carboxylic acid salts of the abovementioned amines and quaternary ammonium salts.

The mixture is heated at a temperature of between 80° C. and 160° C. until the adhesive character of the resultant silicone copolymer is obtained.

The preferred copolymers according to the invention of those having the INCI name Trimethylsiloxysilicate/Dimethiconol Crosspolymer, such as the products sold by Dow Corning under the references Dowsil FC 5002 ID Resin Gum®, Dowsil FC-5001 CM Resin Gum®, Dowsil FC 5004 Resin Gum®, Dowsil FC 5002 ID Resin Gum® (Dow Corning Chemical Company); and more particularly Dowsil FC 5002 ID Resin Gum®/Dow Corning.

The composition according to the invention advantageously has a content of copolymer based on silicone resin and silicone of dimethiconol type ranging from 0.2% to 60% by weight relative to the weight of the composition. Preferably, this content ranges from 1% to 30% by weight relative to the weight of the composition.

More specifically, the copolymer(s) based on silicone resin and on silicone of dimethiconol type may notably be present in the composition according to the invention in a content greater than 1.0% and up to 40% by weight relative to the total weight of the composition, preferably ranging from 1.5% to 20% by weight, and preferentially ranging from 1.5% to 15% by weight.

### Additives

The compositions according to the invention may also comprise additives commonly used in care and/or makeup products, such as lipophilic active agents, hydrophilic active agents such as vitamins, anti-sun UV screening agents, moisturizing agents, for instance polyols such as glycerol, propanediol, pentylene glycol, liposoluble dyestuffs; lipophilic thickeners or gelling agents; hydrophilic thickeners or gelling agents; preservatives, fragrances, and mixtures thereof.

### Pulverulent Dyestuffs

According to one particular form of the invention, the composition according to the invention also comprises at least one pulverulent dyestuff.

The pulverulent dyestuffs can be chosen from mineral pigments, organic pigments, pearlescent agents and mixtures thereof.

The term “pigments” means white or coloured, mineral or organic particles, which are insoluble in an aqueous medium, and which are intended to colour and/or opacify the resulting composition and/or deposit. These pigments may be white or coloured, and mineral and/or organic.

According to a particular embodiment, the pigments used according to the invention are chosen from mineral pigments.

The term “mineral pigment” means any pigment that satisfies the definition in Ullmann's encyclopedia in the chapter on inorganic pigments. Mention may be made, among the mineral pigments which are of use in the present invention, of zirconium oxide or cerium oxide, and also zinc oxide, iron oxide (black, yellow or red) or chromium oxide, manganese violet, ultramarine blue, chromium hydrate and ferric blue, titanium dioxide or metal powders, such as aluminium powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂ or ZnS.

The size of the pigment that is useful in the context of the present invention is generally greater than 100 nm and may range up to 10 μm, preferably from 200 nm to 5 μm and more preferentially from 300 nm to 1 μm.

According to a particular form of the invention, the pigments exhibit a size characterized by a D[50] of greater than 100 nm and which can range up to 10 μm, preferably from 200 nm to 5 μm and more preferentially from 300 nm to 1 μm.

The sizes are measured by static light scattering using a commercial particle size analyser of MasterSizer 3000® type from Malvern, which makes it possible to determine the particle size distribution of all of the particles over a wide range which can extend from 0.01 μm to 1000 μm. The data are processed on the basis of the conventional Mie scattering theory. This theory is the most suitable for size distributions ranging from the submicronic to multimicronic; it makes it possible to determine an “effective” particle diameter. This theory is described in particular in the publication by Van de Hulst, H. C., Light Scattering by Small Particles, Chapters 9 and 10, Wiley, New York, 1957.

D[50] represents the maximum size exhibited by 50% by volume of the particles.

According to a particular form of the invention, the mineral pigment comprises a lipophilic or hydrophobic coating; the latter is preferably present in the oily phase of the composition according to the invention.

According to a particular embodiment of the invention, the pigments may be coated according to the invention with at least one compound chosen from metal soaps; N-acylamino acids or salts thereof; lecithin and derivatives thereof; isopropyl triisostearyl titanate; isostearyl sebacate; natural vegetable or animal waxes; synthetic polar waxes; fatty esters; phospholipids; silicone surfactants such as organopolysiloxanes, alkylalkoxysilanes such as triethoxycaprylylsilane; fluorinated surfactants such as perfluoroalkyl phosphates, perfluoropolyethers, polytetrafluoropolyethylenes (PTFE), perfluoroalkanes, perfluoroalkyl silazanes, poly(hexafluoropropylene oxide) s, polyorganosiloxanes comprising perfluoroalkyl perfluoropolyether groups; fluorosilicone surfactants such as perfluoroalkyl dimethicones, perfluoroalkyl silanes and perfluoroalkyl trialkoxysilanes; and mixtures thereof.

According to a preferential embodiment, the pigments can be coated according to the invention with an N-acylamino acid or one of its salts which can comprise an acyl group having from 8 to 22 carbon atoms, such as, for example, a 2-ethylhexanoyl, caproyl, lauroyl, myristoyl, palmitoyl, stearoyl or cocoyl group.

The amino acid can, for example, be lysine, glutamic acid or alanine. The salts of these compounds can be the aluminium, magnesium, calcium, zirconium, zinc, sodium or potassium salts. Thus, according to a particularly preferred embodiment, the pigments can be coated with an N-acylamino acid derivative which can in particular be a glutamic acid derivative and/or one of its salts, and more particularly a stearoyl glutamate, such as, for example, aluminium stearoyl glutamate. Mention may be made, as examples of pigments treated with aluminium stearoyl glutamate, of titanium dioxide pigments and black, red and yellow iron oxide pigments sold under the trade name Nai® by Miyoshi Kasei.

According to a preferential embodiment, the pigments can be coated according to the invention with isopropyl triisostearyl titanate. Mention may be made, as examples of pigments treated with isopropyl titanium triisostearate (ITT), of those sold under the trade names BWBO-I2® (Iron Oxide CI77499 and Isopropyl Titanium Triisostearate), BWYO-I2® (Iron Oxide CI77492 and Isopropyl Titanium Triisostearate) and BWRO-I2® (Iron Oxide CI77491 and Isopropyl Titanium Triisostearate) by Kobo.

The pigments which can be used according to the invention can also be organic pigments.

The term “organic pigment” refers to any pigment that satisfies the definition in Ullmann's encyclopedia in the chapter on organic pigments. The organic pigment may notably be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) can be chosen, for example, from carmine, carbon black, aniline black, melanin, azo yellow, quinacridone, phthalocyanine blue, sorghum red, the blue pigments codified in the Colour Index under the references CI 42090, 69800, 69825, 73000, 74100 and 74160, the yellow pigments codified in the Colour Index under the references CI 11680, 11710, 15985, 19140, 20040, 21100, 21108, 47000 and 47005, the green pigments codified in the Colour Index under the references CI 61565, 61570 and 74260, the orange pigments codified in the Colour Index under the references CI 11725, 15510, 45370 and 71105, the red pigments codified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as are described in Patent FR 2 679 771.

These pigments can also be in the form of composite pigments as are described in Patent EP 1 184 426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Mention may be made, among the organic dyes, of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

Mention may be made, by way of example of a lake, of the product known under the name D&C Red 7 (CI 15 850:1).

Preferably, the composition according to the invention comprises at least one pulverulent dyestuff of mineral pigment type, in particular chosen from metal oxides, and more particularly chosen from titanium dioxides or iron oxides, which are or are not coated, and mixtures thereof.

The pearlescent agents can be chosen from white pearlescent pigments, such as mica covered with titanium or with bismuth oxychloride, coloured pearlescent pigments, such as titanium oxide-coated mica with iron oxides, titanium oxide-coated mica in particular with ferric blue or chromium oxide, or titanium oxide-coated mica with an organic pigment of the abovementioned type, and also pearlescent pigments based on bismuth oxychloride.

Preferably, the pulverulent dyestuff(s) are preferably present in the composition in a content ranging from 5% to 40% by weight, preferably from 10% to 30% by weight, more particularly from 15% to 25% by weight, relative to the total weight of the composition.

### Liposoluble Dyestuffs

A composition according to the invention may also comprise at least one liposoluble dyestuff, preferably in a proportion of at least 0.01% by weight relative to the total weight of the composition.

Within the meaning of the invention, the term “liposoluble dyestuff” is understood to mean any natural or synthetic, generally organic, compound which is soluble in an oily phase or solvents miscible with a fatty substance and which is capable of imparting colour.

For obvious reasons, this amount is liable to vary significantly with regard to the desired intensity of the colour effect and to the colour intensity provided by the dyestuffs under consideration, and its adjustment clearly falls within the competence of those skilled in the art.

Mention may in particular be made, as liposoluble dyes suitable for the invention, of synthetic or natural liposoluble dyes, such as, for example, DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto or curcumin.

### Water-Soluble Dyestuffs

A composition according to the invention may also comprise at least one water-soluble dyestuff, preferably in a proportion of at least 0.01% by weight relative to the total weight of the composition.

For the purposes of the invention, the term “water-soluble dyestuff” means any natural or synthetic, generally organic compound, which is soluble in an aqueous phase or water-miscible solvents and which is capable of imparting colour.

Mention may in particular be made, as water-soluble dyes suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocyanin, black carrot, hibiscus or elder), caramel or riboflavin.

### Cosmetic Compositions

The present invention also relates to a cosmetic composition comprising, in a physiologically acceptable medium, a composition as defined above.

The term “physiologically acceptable medium” is understood to denote a medium which is particularly suitable for the application of a composition of the invention to the skin.

The physiologically acceptable medium is generally suited to the nature of the support onto which the composition has to be applied, and also to the appearance under which the composition has to be packaged.

The compositions of the invention may be in the form of milks, creams or gels.

### Applications

According to one embodiment, a composition of the invention can advantageously be provided in the form of a composition for caring for the skin of the body or of the face, in particular of the face.

According to another embodiment, a composition of the invention can advantageously be provided in the form of a composition for making up keratin materials, in particular the skin of the body or of the face, in particular of the face.

Thus, according to a sub-mode of this embodiment, a composition of the invention can advantageously be provided in the form of a base composition for makeup.

A composition of the invention can advantageously be provided in the form of a foundation.

Such compositions are in particular prepared according to the general knowledge of those skilled in the art.

Throughout the description, including the claims, the expression “comprising a” should be understood as being synonymous with “comprising at least”.

The invention is illustrated in more detail by the examples and figures presented below. Unless otherwise indicated, the amounts indicated are expressed as mass percentages.

### Examples 2 to 7 (Invention) and Examples 1 and 8 (Outside the Invention)

The following compositions were prepared.

TABLE 1

| | Ex1 | | | | | | | Ex8 |
|---|---|---|---|---|---|---|---|---|
| | outside | | | | | | | outside |
| | the | Ex2 | Ex3 | Ex4 | Ex5 | Ex6 | Ex7 | the |
| Ingredients | invention | invention | invention | invention | invention | invention | invention | invention |
| Isododecane (and) | 10. (i.e. | 2.5 (i.e. | 3 (i.e. | 34 (i.e. | 10 (i.e. | 20 (i.e. | 13 (i.e. | 20 (i.e. |
| Hexyl/Succinyl | 3% of | 0.75% of | 1% of | 10.2% of | 3% of | 6% of | 3.9% of | 6% of |
| Dimethicone | polymer | polymer | polymer | polymer | polymer | polymer | polymer | polymer |
| Crosspolymer | solids) | solids) | active | active | active | active | active | solids) |
| (Dowsil EL-7314 | | | material) | material) | material) | material) | material) | |
| Silicone Elastomer | | | | | | | | |
| Blend ®/Dow Corning | | | | | | | | |
| in gel form in | | | | | | | | |
| dispersion at 30% by | | | | | | | | |
| weight in | | | | | | | | |
| isododecane) | | | | | | | | |
| Isododecane | 24 | 6.5 | 6 | 15 | 9 | 9 | 6 | 3 |
| 75% | 1 | 1 | 1 | 1 | 1 | 1 | 1 | 1 |
| Trimethylsiloxysilicate | | | | | | | | |
| in isododecane | | | | | | | | |
| Distilled water | 65 | 90 | 90 | 50 | 80 | 70 | 80 | 90 |
| [A] = [ratio of the | 0.096 | 0.088 | 0.108 | 0.261 | 0.185 | 0.258 | 0.254 | 0.242 |
| amount of silicone | | | | | | | | |
| elastomer | | | | | | | | |
| containing | | | | | | | | |
| carboxylic acid | | | | | | | | |
| functions relative to | | | | | | | | |
| the total amount by | | | | | | | | |
| weight of volatile | | | | | | | | |
| hydrocarbon oil(s) | | | | | | | | |
| in the composition] | | | | | | | | |
| [B] = [amount of the | 0.65 | 0.90 | 0.90 | 0.50 | 0.80 | 0.70 | 0.80 | 0.90 |
| aqueous phase | | | | | | | | |
| relative to the total | | | | | | | | |
| weight of the | | | | | | | | |
| composition] | | | | | | | | |
| [A] × [B] | 0.06 | 0.08 | 0.10 | 0.13 | 0.15 | 0.18 | 0.20 | 0.22 |

### Protocol for Preparation of the Compositions

The productions of Examples 1 to 8 were carried out with a Trilab® or Olsa Minilab® type reactor from VMI.

The temperature was adjusted to 20° C. in order to maintain a production temperature of 20° C.

The elastomer was pre-dispersed under shearing.

The isododecane was then gradually added to the elastomer until a homogeneous composition was obtained. Trimethylsiloxysilicate was then added, forming the fatty phase.

The macroscopically homogeneous mixture of the phases was prepared by slow introduction of the aqueous phase into the fatty phase at a stirring speed of 4000 rpm for 15 minutes.

The formula was subsequently degassed under a vacuum bell.

### Evaluation of the Stability of the Compositions

All the compositions of Examples 1 to 8 were packaged in a 30 ml glass jar at ambient temperature (25° C.). The stability was observed visually and evaluated at 24 h in accordance with the following criteria:

Stable: The composition is homogeneous without visible phase separation.

Unstable: The composition is not homogeneous (visible phase separation).

The results are described below:

TABLE 2

| | Ex1 | | | | | | | Ex8 |
|---|---|---|---|---|---|---|---|---|
| | outside | | | | | | | outside |
| Stability at 24 | the | Ex2 | Ex3 | Ex4 | Ex5 | Ex6 | Ex7 | the |
| hours | invention | invention | invention | invention | invention | invention | invention | invention |
| Appearance | Unstable | Stable | Stable | Stable | Stable | Stable | Stable | Unstable |
| [A] × [B] | 0.06 | 0.08 | 0.10 | 0.13 | 0.15 | 0.18 | 0.20 | 0.22 |

Examples 2 to 7 according to the invention, having a product [A]×[B] ranging from 0.07 to 0.21, made it possible to obtain a pleasant texture, which can easily be taken up and spread on the skin, with a fresh and light sensation during application and good stability over time.

Examples 1 and 8 outside the invention, having a product [A]×[B] outside the range extending from 0.07 to 0.21, experienced phase separation after 24 hours and were considered to be unstable.

1. Composition for caring for and/or making up keratin materials, in particular the skin, comprising, preferably in a physiologically acceptable medium:
a) an oily phase comprising at least one volatile hydrocarbon oil H₁; and
b) an aqueous phase; and
c) at least one silicone elastomer containing carboxylic acid functions;
said composition having a product of the weight ratios [A]×[B] ranging from 0.07 to 0.21 with
[A] denoting the weight ratio of the amount of silicone elastomer relative to the total weight of the composition to the total amount of volatile hydrocarbon oil(s) relative to the total weight of the composition;
[B] denoting the weight ratio of the amount of aqueous phase relative to the total weight of the composition;
said oily and aqueous phases forming therein a macroscopically homogeneous mixture.

2. Composition according to claim 1, in which the at least one volatile hydrocarbon oil H₁ is chosen from C₈-C₁₆ isoalkanes.

3. Composition according to claim 1, in which the at least one volatile hydrocarbon oil H₁ is chosen from volatile linear alkanes comprising from 6 to 14 carbon atoms.

4. Composition according to claim 1, in which the at least one volatile hydrocarbon oil H₁ is:
a mixture of n-dodecane and of n-tetradecane;
a mixture of volatile linear C₉-C₁₂ alkanes; or
a mixture of n-undecane (C₁₁) and of n-tridecane (C₁₃).

5. Composition according to claim 1, in which the oily phase is present in an amount from 5% to 95% by weight relative to the total weight of the composition.

6. Composition according to claim 1, in which water is present in a concentration from 5% to 95% by weight relative to the total weight of the composition.

7. Composition according to claim 1, in which the aqueous phase is present in a concentration from 5% to 95% by weight relative to the total weight of the composition.

8. Composition according to claim 1, in which the at least one silicone elastomer containing carboxylic acid functions is present in an amount from 2% to 25% by weight, relative to the weight of the composition.

9. Composition according to claim 1, in which a weight ratio of the total amount of volatile hydrocarbon oil(s) relative to the total weight of the composition to the amount of aqueous phase relative to the total weight of the composition is greater than 0.085.

10. Composition according to claim 1, in which the at least one silicone elastomer containing carboxylic acid functions is chosen from at least one of the compounds corresponding to the formula (I) below: 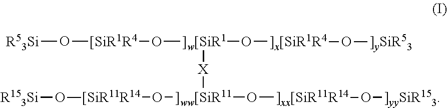
in which the radical X corresponds to formula (i) or formula (i′) below: 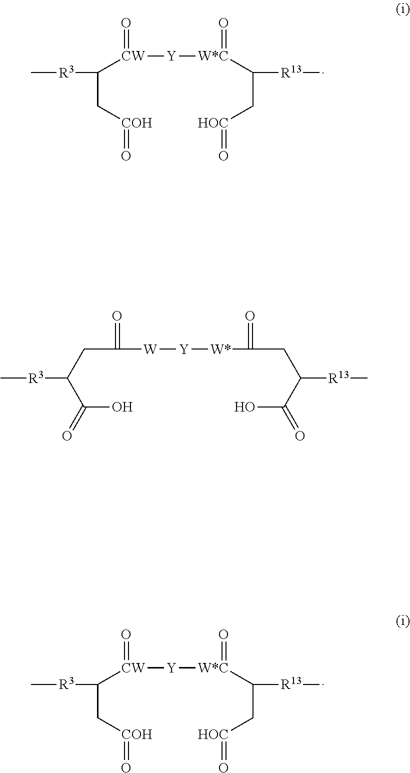
in which
W and W* independently denote an oxygen atom (O) or an N—R group where each R radical independently denotes a hydrogen atom (H) or an R¹ radical;
Y is a divalent group;
R¹, R¹¹, R⁴, R¹⁴, R⁵ and R¹⁵ independently denote a substituted or unsubstituted hydrocarbyl group;
R³ and R¹³ independently denote a divalent group;
w and ww are independently integers ranging from 0 to 1000;
x and xx are, independently, integers ranging from 1 to 100; and
y and yy are, independently, integers ranging from 0 to 1000.

11. Composition according to claim 1, in which the at least one silicone elastomer containing carboxylic acid functions is chosen from at least one of the following compounds 1 to 12:

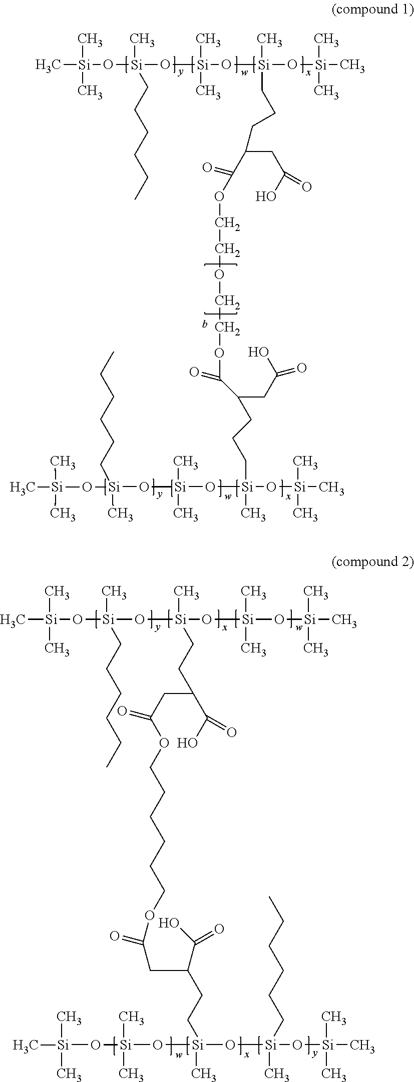

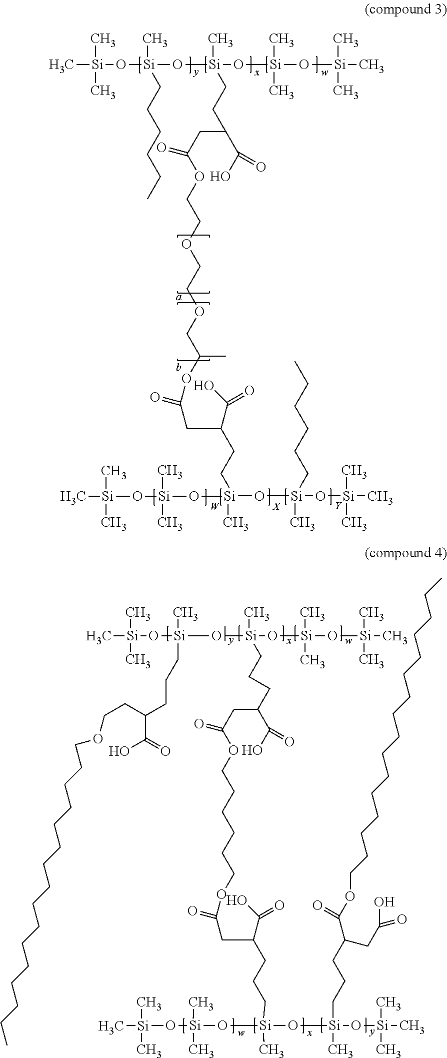

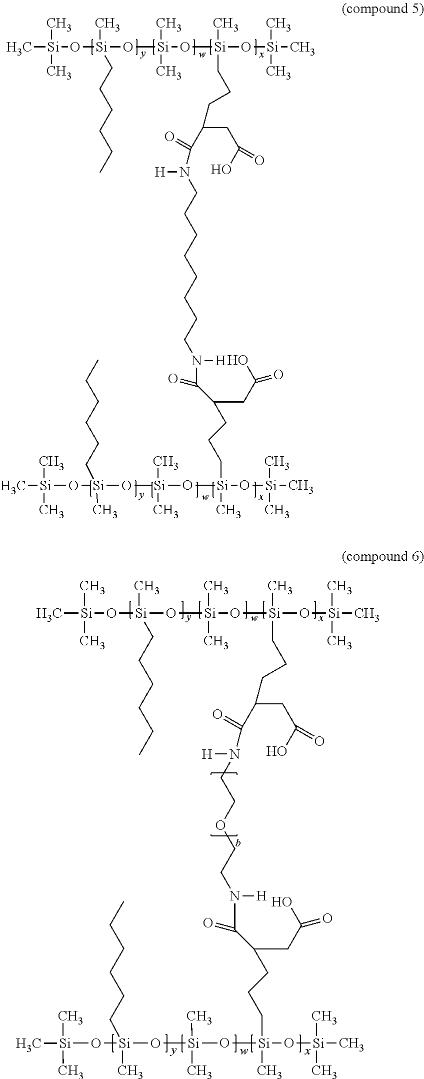

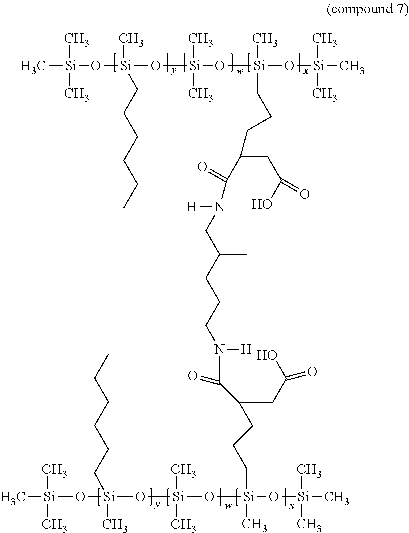

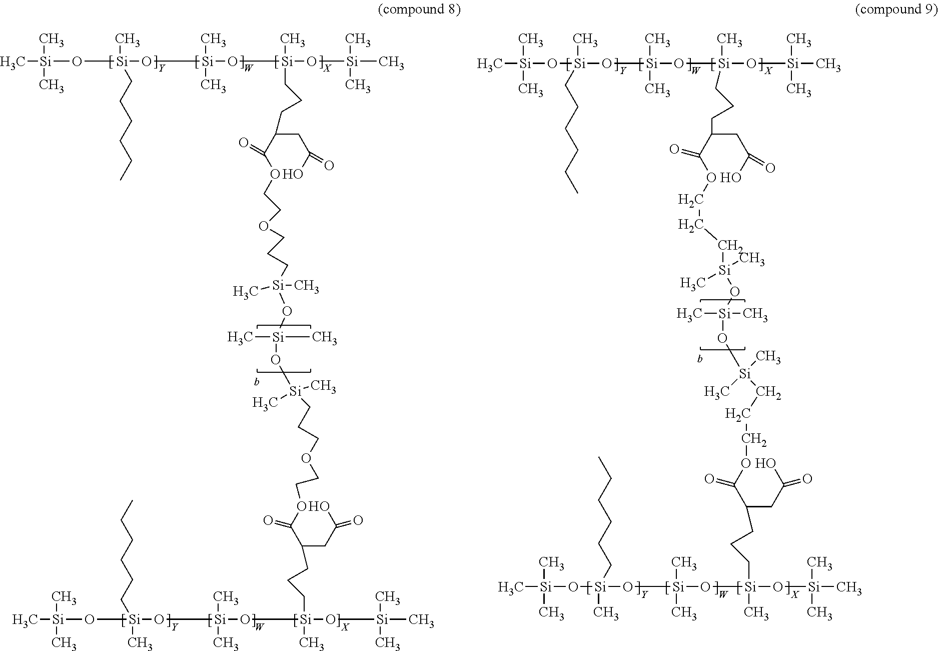

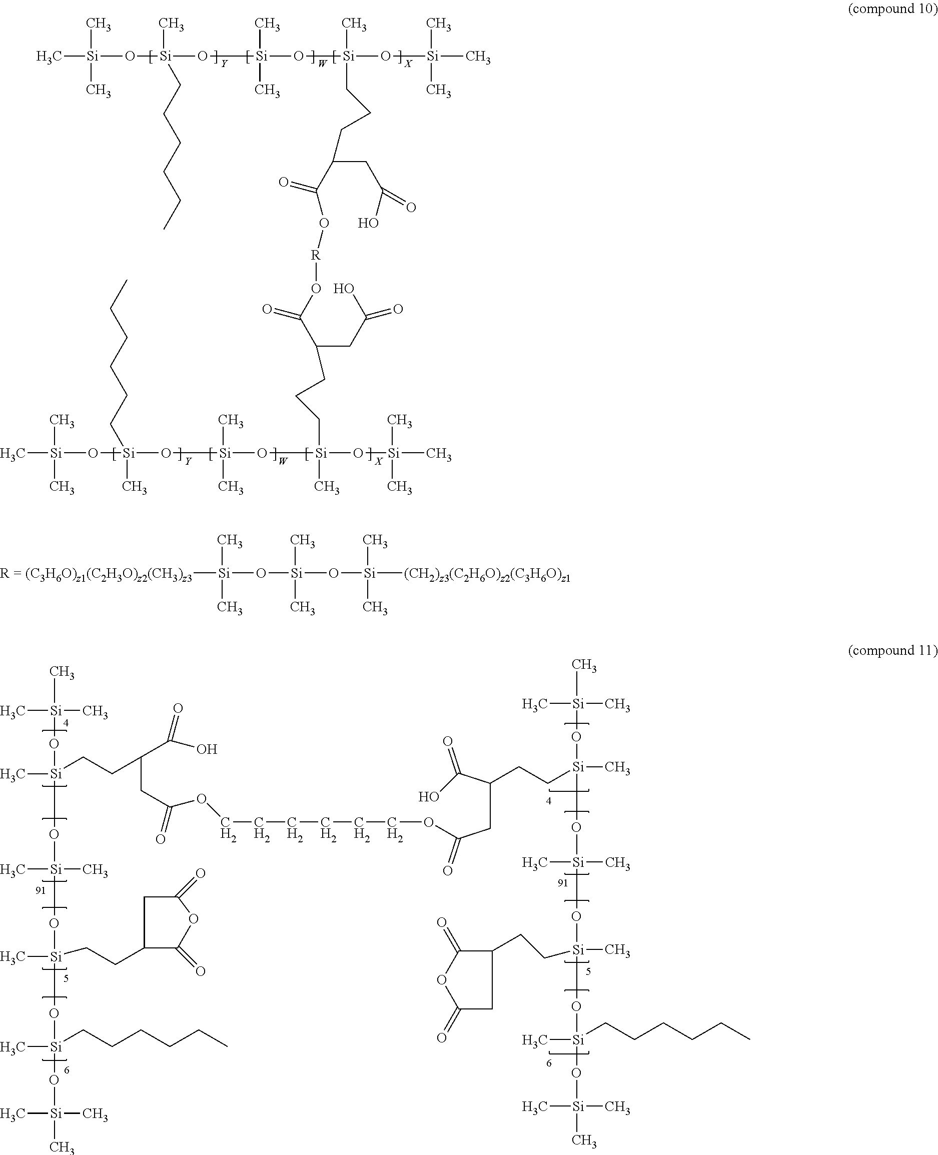

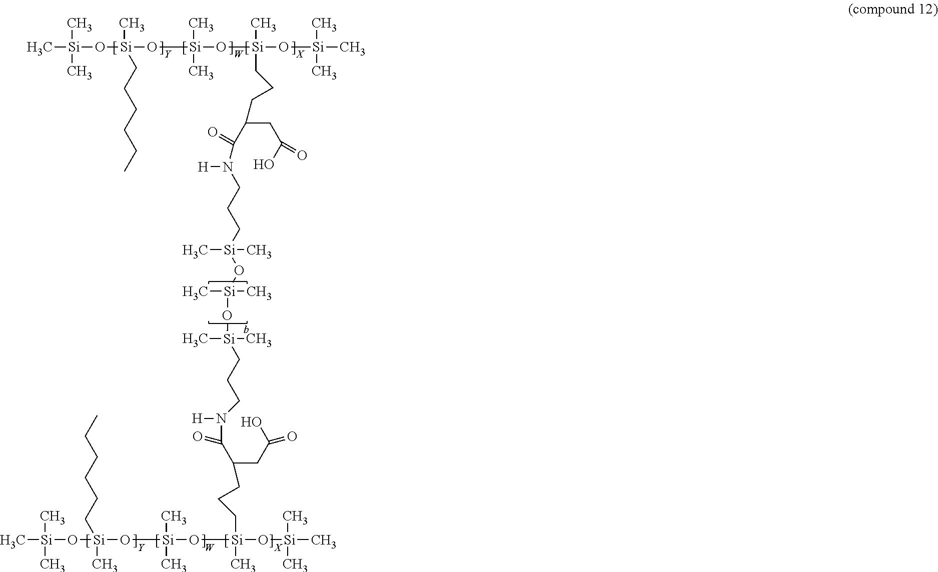

12. Composition according to claim 11, in which the at least one silicone elastomer containing carboxylic acid functions is compound 10 and/or compound 11.

13. Composition according to claim 1, in which the at least one silicone elastomer containing carboxylic acid functions is Hexyl/Succinyl Dimethicone Crosspolymer.

14. Composition according to claim 1, in which the at least one silicone elastomer containing carboxylic acid functions is in dispersion in at least one oil H₂ and is in gel form.

15. Composition according to claim 14, in which the at least one oil H₂ is a volatile hydrocarbon oil chosen from C₈-C₁₆ isoalkanes and linear alkanes comprising from 6 to 14 carbon atoms.

16. Composition according to claim 14, in which the at least one oil H₂ and the at least one oil H₁ are identical.

17. Composition according to claim 14, in which the dispersion of silicone elastomer containing carboxylic acid functions comprises from 10% to 40% by weight of silicone elastomer active material relative to the total weight of the dispersion.

18. Composition according to claim 14, in which the at least one silicone elastomer containing carboxylic acid functions is in dispersion in isododecane.

19. Composition according to claim 12, in which the at least one silicone elastomer containing carboxylic acid functions is present in isododecane in an active material content of 30% by weight with respect to the total weight of the dispersion.

20. Composition according to claim 13, in which the Hexyl/Succinyl Dimethicone Crosspolymer is in the form of a gel in dispersion in isododecane.

21. Composition according to claim 1, also comprising at least one silicone resin and/or at least one copolymer of silicone resin and silicone of dimethiconol.

22. Composition according to claim 21, in which the at least one silicone resin is present and is an MQ resin.

23. Composition according to claim 21, in which the copolymer of silicone resin and silicone of dimethiconol is present and is Trimethylsiloxysilicate/Dimethiconol Crosspolymer.

24. Composition according to claim 1, also comprising at least one additive chosen from anti-sun UV screening agents, C₂-C₄ monoalcohols, such as ethanol, moisturizing agents, pulverulent dyestuffs, liposoluble dyestuffs, lipophilic thickeners or gelling agents, preservatives, fragrances, and mixtures thereof.

25. Process for coating keratin materials, more particularly for making up and/or caring for keratin materials, such as the skin, characterized in that it comprises the application to the keratin materials of a composition as defined in claim 1.
